(19) United States
(12) Patent Application Publication
(10) Pub. No.: US 2024/0173530 A1
(43) Pub. Date: May 30, 2024

(54) Disinfecting Cap For Male Connectors

(71) Applicant: Becton, Dickinson and Company

(72) Inventors: Shishir Prasad, Ramsey, NJ (US); Karthik MR, Bangalore (IN); Manish Kumar, Bengaluru (IN); Balaji Kannan, Chennai (IN)

(21) Appl. No.: 17/994,903

(22) Filed: Nov. 28, 2022

(51) Int. Cl. A61M 39/16; A61M 39/20

(52) U.S. Cl. CPC A61M 39/162; A61M 39/20; A61M 2207/10

(57) ABSTRACT

A cap configured to engage a male connector includes a housing having an open first end, a second end, and at least one sidewall extending between the first end and the second end. The cap also includes an outer absorbent support disposed in the housing defining a recess and a socket disposed in the recess of the outer absorbent support configured to engage the male connector for retaining the male connector within the housing. The cap also includes an inner absorbent support disposed in the socket configured to contain a cleaning solution for cleaning and/or disinfecting portions of the male connector engaged to the cap.

### BACKGROUND OF THE INVENTION

### Field of the Invention

The present disclosure relates generally to caps for medical connectors and, in particular, to a medical cap configured to be attached to male connectors for sealing, cleaning, and disinfecting portions of the male connectors.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Further, bacteria and other microorganisms may enter into a patient's vascular system from access hubs, ports, or valves upon connection to the VAD to deliver a fluid or pharmaceutical to a patient. Therefore, each access hub, port, valve, or other connection configured for attachment to a VAD is associated with some risk of transmitting a catheter related bloodstream infection (CRBSI) to a patient.

Many medical facilities implement sterile practices and protocols to ensure that VADs and access hubs or ports are used properly and do not become sealed or infected. These protocols often include sterilizing the access hubs, ports, and VADs, as well as flushing the catheter with a flush solution prior to use. Specifically, VAD standards of practice usually recommend flush procedures be performed after catheter placement, before fluid infusion, and before and after drug administration, blood sampling, transfusions, and/or administration of parenteral nutrition.

Standards of practice can also require that access hubs, ports, and valves be capped with disinfection caps when not in use, to prevent microbial ingress into the hub, port, or valve and to sterilize areas of the hub, port, or valve that contact the VAD. Disinfection caps are disposable cap devices that contain an amount of cleaning or disinfecting solution for sterilizing portions of the port, hub, and valve. Accordingly, disinfecting caps can be both a barrier preventing microbes, particles, and debris from contacting portions of hubs, ports, or VADs that should remain sterile and disinfecting or cleaning devices configured to expose portions of the hubs, ports, and VADs to cleaning and disinfecting solutions.

However, conventional disinfecting caps may only clean or disinfect distal-most portions of a male connector, such as a tip of a luer or stem. Conventional caps may not clean and disinfect other surfaces of the male connector in order to ensure that an entire distal region of the male connector remains sterile. The disinfecting caps of the present disclosure are configured to address these issues.

### SUMMARY OF THE INVENTION

According to an aspect of the disclosure, a cap configured to engage a male connector includes a housing having an open first end, a second end, and at least one sidewall extending between the first end and the second end. The cap also includes an outer absorbent support disposed in the housing defining a recess and a socket disposed in the recess of the outer absorbent support configured to engage the male connector for retaining the male connector within the housing. The cap also includes an inner absorbent support disposed in the socket configured to contain a cleaning solution for cleaning and/or disinfecting portions of the male connector engaged to the cap.

In accordance with an embodiment of the present invention, the male connector includes a male luer connector.

In accordance with an embodiment of the present invention, the male luer connector includes a luer stem defining a central lumen and a shield extending about the stem.

In accordance with an embodiment of the present invention, the shield of the male luer connector includes threads on an inner surface of the shield configured to engage corresponding threads of a female luer connector for securing the male connector to the female luer connector.

In accordance with an embodiment of the present invention, when the male luer connector is engaged to the cap, the luer stem of the male connector is inserted into the socket and an outer surface of the shield is in contact with an inner surface of the outer absorbent support.

In accordance with an embodiment of the present invention, when the male connector is engaged to the cap, cleaning solution is exposed to the threads on the inner surface of the shield for cleaning and/or disinfecting the threads.

In accordance with an embodiment of the present invention, the cap is sized to receive male connectors having different thread configurations and dimensions.

In accordance with an embodiment of the present invention, the housing includes a rigid thermoplastic polymer having at least one of polyester, polycarbonate, polypropylene, polyethylene, polyethylene terephthalate, or acrylonitrile butadiene styrene.

In accordance with an embodiment of the present invention, the housing includes a plurality of ridges or ribs extending outwardly from an outer surface of the housing (for making the housing easier to grip).

In accordance with an embodiment of the present invention, an inner surface of the sidewall of the housing includes an inwardly sloping tapered portion, and wherein an outer surface of the outer absorbent support comprises an angled portion matching the tapered portion of the sidewall.

In accordance with an embodiment of the present invention, the housing includes a first part, which includes the open first end of the housing, formed from a flexible material, and a second part, which includes the closed second end of the housing, formed from a rigid material.

In accordance with an embodiment of the present invention, the first material includes a thermoplastic elastomer having at least one of silicone, polypropylene, polyethylene, or synthetic or natural rubber (e.g., isoprene) and the second material comprises a rigid thermoplastic polymer, having at least one of polyester, polycarbonate, polypropylene, polyethylene, polyethylene terephthalate, or acrylonitrile butadiene styrene.

In accordance with an embodiment of the present invention, the first part includes a tubular member having an open top, an open bottom, and a sidewall extending between the top and the bottom, and the second part includes an open top adhered to the open bottom of the first part, a closed bottom, and a sidewall extending between the top and the bottom.

In accordance with an embodiment of the present invention, further including a knob extending from the second end of the housing for grasping the housing.

In accordance with an embodiment of the present invention, the outer absorbent support is axially compressible.

In accordance with an embodiment of the present invention, the recess defined by the outer absorbent support includes a first portion having a first diameter corresponding to an outer diameter of an annular shield of the male connector and a second portion axially aligned with the first portion having a second diameter corresponding to an outer diameter of the socket.

In accordance with an embodiment of the present invention, the first diameter is larger than the second diameter.

In accordance with an embodiment of the present invention, the outer absorbent support includes an annular member that is axially compressible and encloses a central cylindrical recess.

In accordance with an embodiment of the present invention, the outer absorbent support includes a thermoplastic elastomer having at least one of silicone, polypropylene, polyethylene, or synthetic or natural rubber (e.g., isoprene).

In accordance with an embodiment of the present invention, the socket includes an open first end, a second end, and a sidewall extending between the first end and the second end.

In accordance with an embodiment of the present invention, the socket further includes a thread extending radially outward from the sidewall of the socket, the thread configured to engage threads on an inner surface of a shield of the male connector.

In accordance with an embodiment of the present invention, the engagement between the thread of the socket and the threads of the shield of the male connector rotatably secure the male connector within the housing.

In accordance with an embodiment of the present invention, the sidewall of the socket includes at least one opening for permitting the cleaning solution to pass through the socket to the outer absorbent support.

In accordance with an embodiment of the present invention, the at least one opening includes a slot extending axially from the open first end of the socket.

In accordance with an embodiment of the present invention, the at least one opening includes a first slot extending axially from the open first end of the socket and a second slot extending axially from the open first end of the socket on an opposite side of the socket from the first slot.

In accordance with an embodiment of the present invention, the second end of the socket is adhered to the second end of the housing, thereby securing the socket within the housing.

In accordance with an embodiment of the present invention, the open first end of the socket is entirely enclosed in the housing.

In accordance with an embodiment of the present invention, the socket includes a rigid thermoplastic polymer, having at least one of polyester, polycarbonate, polypropylene, polyethylene, polyethylene terephthalate, or acrylonitrile butadiene styrene.

In accordance with an embodiment of the present invention, the socket is formed from a same rigid thermoplastic polymer as the housing.

In accordance with an embodiment of the present invention, the inner absorbent support includes a thermoplastic elastomer comprising at least one of silicone, polypropylene, polyethylene, or synthetic or natural rubber (e.g., isoprene).

In accordance with an embodiment of the present invention, the inner absorbent support includes a sponge.

In accordance with an embodiment of the present invention, the inner absorbent support includes an open cell foam, such as a porous foam comprising a thermoplastic elastomer.

In accordance with an embodiment of the present invention, wherein insertion of the male connector into the socket causes the inner absorbent support to axially compress farther into the socket.

In accordance with an embodiment of the present invention, the axial compression of the inner absorbent support expels the cleaning solution from the inner absorbent support causing the cleaning solution to move through an interior of the socket contacting an outer surface of a stem of the male connector inserted in the socket.

In accordance with an embodiment of the present invention, the axial compression of the inner absorbent support causes the cleaning solution of the inner absorbent support to pass through the socket and to be absorbed by the outer absorbent support.

In accordance with an embodiment of the present invention, the cleaning solution is absorbed by the inner absorbent support.

In accordance with an embodiment of the present invention, the cleaning solution includes Isopropyl Alcohol (IPA).

In accordance with an embodiment of the present invention, the cleaning solution includes from about 0.5% to about 3.5% chlorhexidine gluconate and about 70% IPA

In accordance with an embodiment of the present invention, a seal is disposed in the socket over the inner absorbent support configured to seal an open end of the male connector, thereby preventing the cleaning solution from entering a lumen of the male connector.

In accordance with an embodiment of the present invention, the seal includes a non-porous foam, such as a closed cell foam.

In accordance with an embodiment of the present invention, the seal is positioned to permit the cleaning solution expelled from the inner absorbent member to move past the seal towards the first end of the housing when the inner absorbent member is axially compressed.

In accordance with an embodiment of the present invention, an outer diameter of the seal is less than an outer diameter of the inner absorbent support and/or an inner diameter of the socket.

In accordance with an embodiment of the present invention, the seal includes at least one of holes, cut-away portions, grooves, slits, or slots for permitting the cleaning solution to move past the seal towards the open first end of the housing.

In accordance with an embodiment of the present invention, a layer of the cleaning solution is disposed over a top surface of the seal.

In accordance with an embodiment of the present invention, a protective cover is provided over the open first end of the housing.

In accordance with an embodiment of the present invention, the protective cover is attached to the housing by heat sealing.

According to another aspect of the disclosure, a method for attaching the previously described cap to the male connector includes a step of inserting a distal end of a stem of the male connector through the open first end of the housing causing the distal end of the stem to contact a seal disposed in the socket over the inner absorbent support. The method also includes a step of rotating the male connector relative to the housing causing the stem of the male connector to advance through the socket axially compressing the inner absorbent support.

In accordance with an embodiment of the present invention, the method includes inserting a distal end of a stem of the male connector through the open first end of the housing causing the distal end of the stem to contact a seal disposed in the socket over the inner absorbent support; and rotating the male connector relative to the housing causing the stem of the male connector to advance through the socket axially compressing the inner absorbent support.

In accordance with an embodiment of the present invention, the method includes continuing to rotate the male connector relative to the housing causing a shield of the male connector to press against and compress the outer annular support.

In accordance with an embodiment of the present invention, the method includes advancing the stem of the male connector through the socket causes the cleaning solution to be expelled from the inner absorbent support and to move through the socket contacting an outer surface of the stem of the male connector.

In accordance with an embodiment of the present invention, contact between the distal end of the stem and the seal seals a distal opening of the stem preventing the cleaning solution from passing into a lumen of the male connector.

In accordance with an embodiment of the present invention, upon axial compression of the inner absorbent support, the cleaning solution moves past the seal towards the open first end of the housing contacting an outer surface of the stem of the male connector.

According to another aspect of the disclosure, a manufacturing method for the previously described cap includes steps of inserting the inner absorbent support into the socket and inserting the socket and inner absorbent support inserted therein into the recess of the outer absorbent support. The method also includes a step of forming the housing about an outer surface of the outer absorbent support by over-molding.

In accordance with an embodiment of the present invention, the method further includes inserting the inner absorbent support into the socket and inserting the socket and inner absorbent support inserted therein into the recess of the outer absorbent support; and forming the housing about an outer surface of the outer absorbent support by over-molding.

In accordance with an embodiment of the present invention, the housing includes a rigid thermoplastic polymer having at least one of polyester, polycarbonate, polypropylene, polyethylene, polyethylene terephthalate, or acrylonitrile butadiene styrene.

In accordance with an embodiment of the present invention, the method includes forming the housing by forming a bottom part of the housing from a rigid thermoplastic polymer and forming a top part of the housing from a thermoplastic elastomer.

### BRIEF DESCRIPTION OF THE DRAWINGS

FIG. 1A is a cross-sectional view of an exemplary male connector, as is known in the prior art.

FIG. 1B is an example of a closed female connector including a septum with a slit, as is known in the prior art.

FIG. 2A is a perspective view of a disinfecting cap for a male connector, according to an aspect of the present disclosure.

FIG. 2B is a cross-sectional view of the cap of FIG. 2A.

FIG. 2C is an exploded perspective view of the cap of FIG. 2A.

FIG. 3A is a perspective view of a housing of the cap of FIG. 2A, according to an aspect of the present disclosure.

FIG. 3B is a cross-sectional view of the housing of FIG. 3A.

FIG. 4A is a cross-sectional view of the cap of FIG. 2A and a male connector, prior to inserting the male connector into the cap.

FIG. 4B is a cross-sectional view of the cap of FIG. 2A with the male connector partially inserted into the cap.

FIG. 4C is a cross-sectional view of the cap of FIG. 2A with the male connector fully inserted into the cap.

FIG. 5 is a flow chart showing a method for attaching the disinfecting cap to a male connector, according to an aspect of the present disclosure.

FIG. 6A is a perspective view of another example of a disinfecting cap for a male connector, according to an aspect of the disclosure.

FIG. 6B is a cross-sectional view of the cap of FIG. 6A.

FIG. 7A is a cross sectional view of the cap of FIG. 6A and a male connector, prior to inserting the male connector into the cap.

FIG. 7B is a cross-sectional view of the cap of FIG. 6A with the male connector fully inserted into the cap.

FIG. 8A is a front view showing another example of a disinfecting cap for male connectors, prior to inserting the male connector into the cap.

FIG. 8B is a cross-sectional view of the cap of FIG. 8A with the male connector fully inserted into the cap.

FIG. 9A is a perspective view of a distal end of a male connector showing a seal of a disinfecting cap covering an open end of a lumen of the male connector, according to an aspect of the present disclosure.

FIG. 9B is a perspective view of the seal of FIG. 9A.

FIG. 10A is a perspective view of another exemplary seal that can be used with a disinfecting cap for a male connector, according to an aspect of the present disclosure.

FIG. 10B is a perspective view of another exemplary seal that can be used with a disinfecting cap for a male connector, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. The term “proximal” refers to a portion of a device or part that is grasped by a user or connected to another device or part. The term “distal” refers to a portion of a device or part that is opposite the proximal portion (i.e., farthest away from the portion that is grasped by a user or connected to another part). For example, for an implantable medical device, such as a catheter, a proximal portion can refer to the portion of the catheter that remains outside of a patient's body and is manipulated by a user. The distal portion of the catheter can be the portion that is inserted into the vasculature of the patient. For an object or part, such as an elongated member, the proximal end can be the end that is connected to another object or part. The distal end of the elongated member is opposite the proximal end and can be free from connections to other objects or parts. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a disinfecting cap 10 configured to be connected to a male connector 110, such as a male connector of an access hub, port, or valve for a VAD, to prevent the connector, port, or VAD from being contaminated by, for example, microbes, debris, or other contaminants. For example, the disinfecting cap 10 can be a male disinfecting cap for disinfecting ISO594-2 type of female threaded fluid luer connectors. In some examples, the cap 10 can be configured to clean or disinfect portions of the connector 110 or port, ensuring that the connector 110 or port remains sterile prior to use. The cap 10 can be configured to remain in place on a connector 110 or port for at least seven days, which is a maximum time of recommended use permitted by many medical facility sterile practice guidelines.

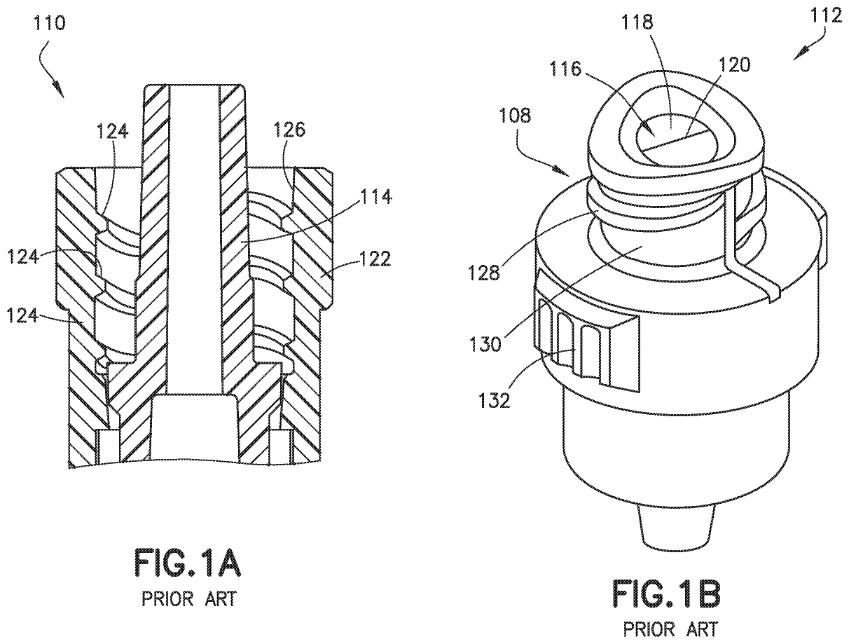

The cap 10 is a disinfecting cap for male connectors configured to engage with or be connected to different sizes, configurations, and/or types of male connectors 110. As used herein, a “male connector” refers to a connector 110 comprising an elongated member, such as a tubular member or stem 114, configured to be inserted in a tube or opening having an inner diameter that is larger than an outermost diameter of the male connector 110. An exemplary male connector 110 is shown in FIG. 1A. The male connector 110 is configured to be connected to a hub, port, or another portion of a VAD comprising a female connector. As used herein, the “female connector” refers to a connector 112 comprising an opening or port 116 that is configured to receive an elongated member or tubular member of another object or device in order to connect the object or device to the female connector 112. The female connector 112 can comprise an elongated distal end portion 108 with a cover or septum 118 over the opening 116. An exemplary female connector 112 including a septum 118 with a slit 120 is shown in FIG. 1B.

In some examples, the cap 10 can be configured to be connected to or engage different types (e.g., different sizes and shapes) of male luer connectors. For example, the cap 10 can be sized to receive different types of male luer connector 110 having an outer diameter of from about 8.0 mm to about 12.0 mm. As used herein, a “luer connector” refers to a connector that includes a tapered portion (i.e., a luer taper) for creating a friction engagement between a tapered stem 114 or elongated member of a male luer connector 110 and a tapered cavity. For example, the male luer connector 110 can include a tapered stem 114 or elongated member having a tapered outer surface. A female luer connector 112 configured to engage the male luer connector 110 can include a tapered cavity configured to receive and engage the tapered stem 114 or elongated member to connect the male luer connector 110 to the female luer connector 112.

In some examples, the male connectors 110 can also include engaging structures, such as threads, for drawing the male connectors 110 into or against a female port or hub. For example, as shown in FIG. 1A, the male luer connector 110 can include an annular shield 122 extending about the tapered stem 114 or elongated member. The annular shield 122 can include threads 124 on an inner surface 126 of the shield 122 configured to engage corresponding threads 128 on an outer surface 130 of the female luer connector 112. Specifically, as shown in FIG. 1B, the female luer connector 112 can include threads 128 extending from the outer surface 130 positioned to engage the threads 124 on the inner surface 126 of the annular shield 122 of the male luer connector 110. Twisting the female connector 112 relative to the male connector 110 causes the corresponding threads 124, 128 to engage, which draws the connectors 110, 112 together, such that the tapered stem 114 or elongated member of the male luer connector 110 moves through the opening 116 of the female connector 112. In some examples, the female connector 112 can also include vertical ribs 132 near a proximal end of the female connector 112, which can be used to manipulate the female connector 112 making it easier to twist the female connector 112 relative to another connector or device.

There are numerous commercially available medical devices, such as hubs, ports, and valves, which include different variations of male connectors 110, such as male luer connectors. As described in further detail herein, in some examples, the cap 10 of the present disclosure includes a flexible housing that can stretch, bend, or otherwise deform in order to engage and securely connect to different types and sizes of male connectors 110. For example, the cap 10 can be configured to attach to a male Luer-Lok™ connector by Becton Dickinson and Company. The cap 10 can also be configured to cover different connector designs including, without limitation, the BD Q-Syte™, BD MaxZero™, BD MaxPlus™, and SmartSite™ needle free connectors by Becton Dickinson and Company. The cap 10 can also be configured to be connected to male connectors by other manufactures including, without limitation, MicroClave® connectors (ICU Medical Inc.) and Ultrasite® connectors (B. Braun Medical Inc.). In other examples, the cap 10 can be configured to connect to one or more of the following commercially available male connectors: Kendall 2001NP; BD MP5303-C; ICU Med 12664-28; RyMed RYM-5307HPU; B. Braun 470108; Baxter 2C8537; Kawasumi IV-0094; Zyno B2-70071-D; B. Braun 470124; Baxter 2C7462; and Smith's Medical 536035.

### Disinfecting Cap for Male Connectors

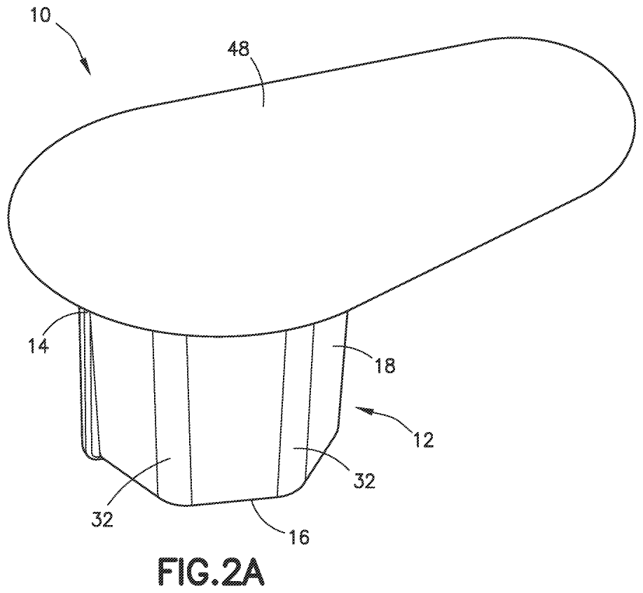

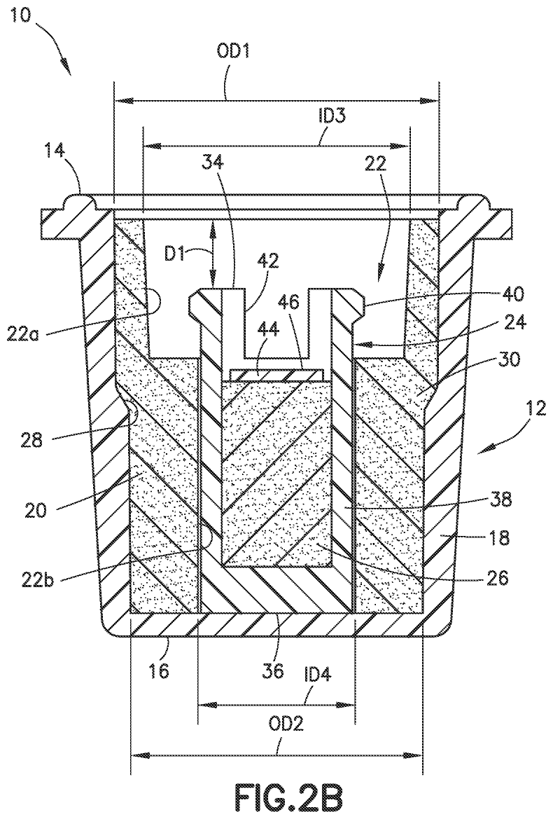

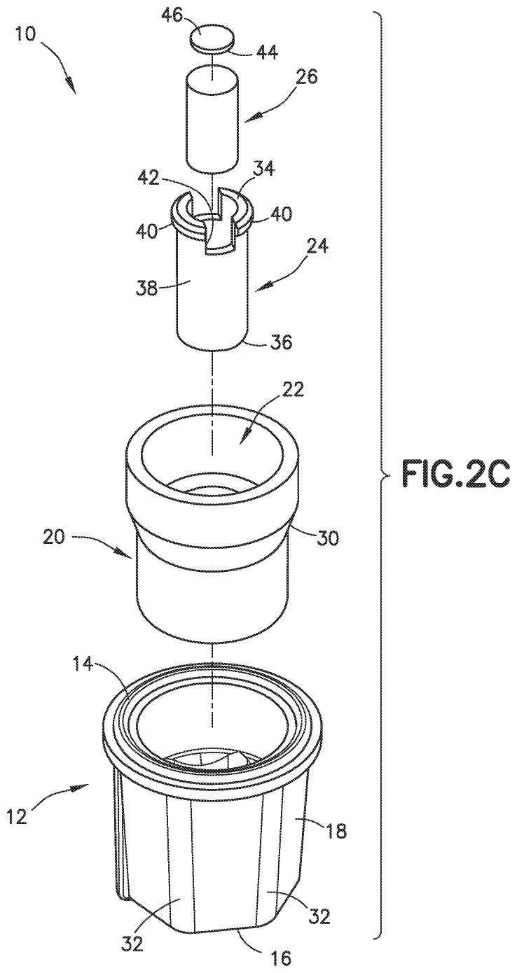

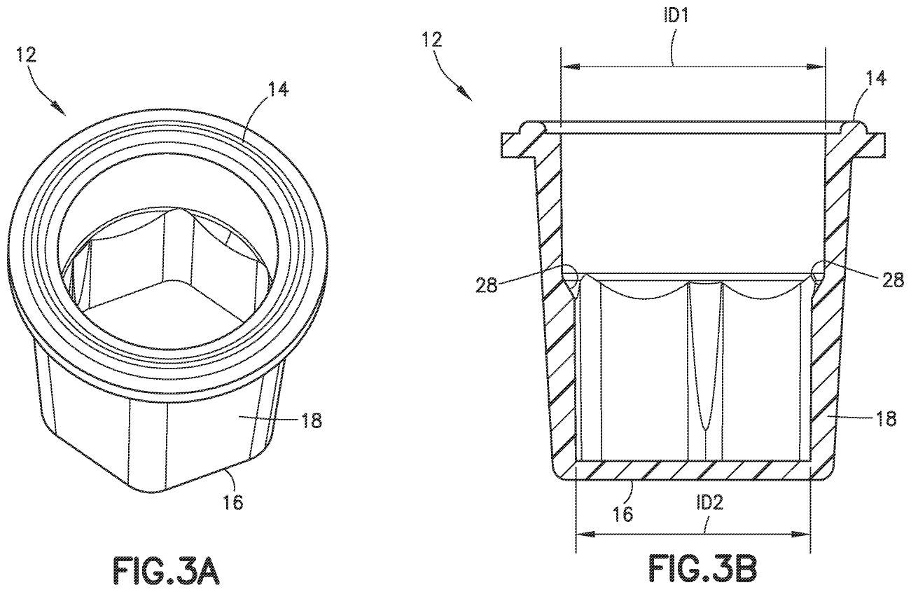

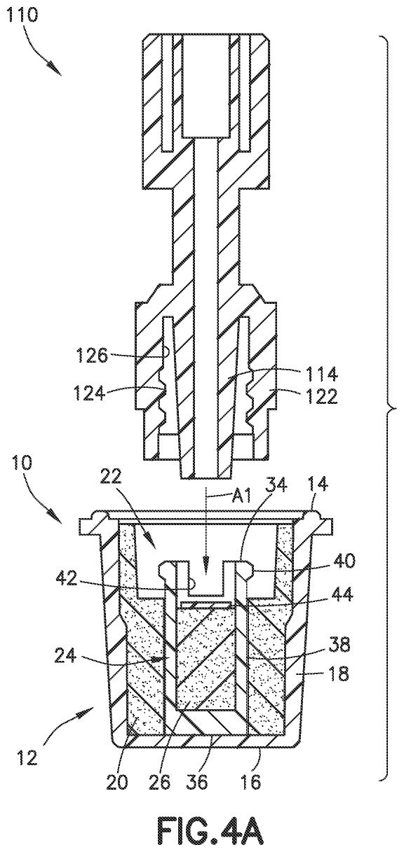

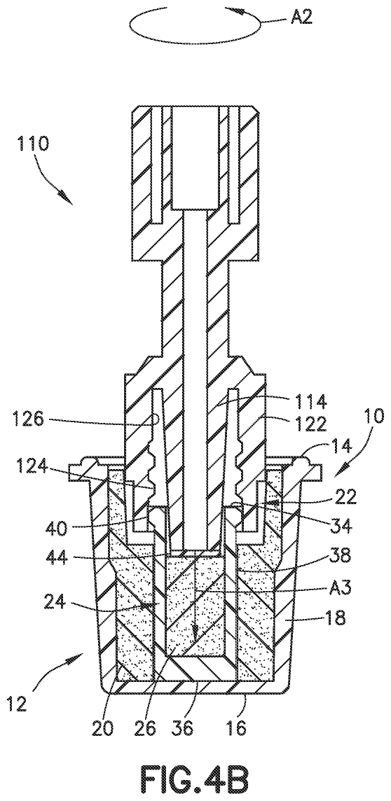

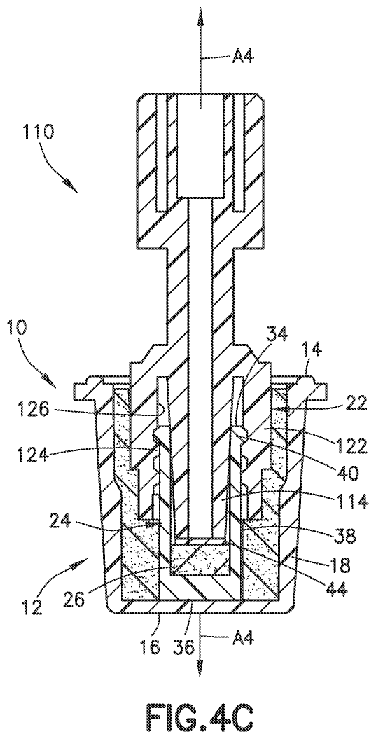

FIGS. 2A-4C illustrate portions of an exemplary disinfecting cap 10 configured to engage and/or to be connected to male connectors 110 with different shapes, sizes, dimensions, or configurations. Specifically, FIG. 2A is a perspective view of the cap 10 prior to use with a protective cover adhered over an open top or first end of the cap 10. FIG. 2B is a cross-sectional view of the cap 10 after the protective cover is removed. FIG. 2C is an exploded view of the cap 10. FIG. 3A is a perspective view of a housing of the cap 10 showing an open top or first end of the housing. FIG. 3B is a cross-sectional view of the housing 12. FIGS. 4A-4C are cross-sectional views showing the male connector 110 being inserted into the disinfecting cap 10.

In some examples, the cap 10 can be provided as a single pre-packaged cap or cap assembly, such as the packaged cap shown in FIG. 2A. Further, as described in detail herein, the cap 10 includes components, such as sponges, abrasive surfaces, and/or cleaning or disinfecting solutions, for cleaning, scrubbing, and disinfecting portions of the male connectors 110 inserted into and mounted to the cap 10.

As shown in FIGS. 2A-4C, the cap 10 comprises a housing 12 comprising an open first end or top 14, a second or bottom 16, and a sidewall 18 extending between the top 14 and the bottom 16. The cap 10 further comprises an outer absorbent support 20 disposed in the housing 12. The outer absorbent support 20 can be a tubular member defining a central recess 22 or cavity sized to receive portions of the male connector 110. The cap 10 also comprises a socket 24 disposed in the recess 22 of the outer absorbent support 20 configured to engage the male connector 110 for retaining the male connector 110 within the housing 12. For example, the stem 114 of the male connector 110 can be inserted into the socket 24 and threads 124 on the inner surface 126 of the shield 122 can engage corresponding threaded structures of the socket 24 for securing the male connector 110 to the socket 24. The cap 10 also includes cleaning parts, such as sponges, brushes, or porous members, disposed in the socket 24, for cleaning distal portions of the male connector 110. For example, the cap 10 can comprise an inner absorbent support 26 disposed in the socket 24 configured to contain a cleaning solution for cleaning and/or disinfecting portions of the male connector 110 engaged to the cap 10.

The outer absorbent support 20, inner absorbent support 26, and other parts of the cap 10 of the present disclosure are configured to cause the cleaning solution to contact multiple areas of the male connector 110 ensuring that the multiple areas of the male connector 110 remain sterile and free from microbes, contaminants, particles, and other debris. In particular, the cap 10 is configured to ensure that cleaning solution contacts not only a distal end or tip of the stem 114 of the male connector 110, but also outer surfaces of the stem 114, as well as inner and outer surfaces of the annular shield 122. Importantly, the cleaning solution should contact both roots and crests of the threads 124 on the inner surface 126 of the shield 122 to ensure that the threads 124 remain clean, sterile, and ready for use.

In some examples, the housing 12 is a molded part formed by injection molding or other common molding processes. The housing 12 can also be formed by an over-molding method in which the housing 12 is molded about the outer surface of the outer absorbent support 20 after the other components of the cap 10 (e.g., the socket 24, inner absorbent support 26, and outer absorbent support 20) are assembled together. In some examples, the housing 12 can be formed from a rigid thermoplastic polymer material, such as polyester, polycarbonate, polypropylene, polyethylene, polyethylene terephthalate, or acrylonitrile butadiene styrene. Further, the housing 12 can be formed from a durable material, such as a material having a shore hardness D value of less than or equal to 95 (Shore D). Alternatively, the housing 12 can be formed from a more flexible material, such as a material having a shore hardness A value less than or equal to 130 (Shore A). Beneficially, a flexible housing 12 or housing 12 including flexible portions can be capable of stretching, bending, or otherwise deforming so that the cap 10 is capable of engaging male connectors 110 of different sizes and shapes.

In some examples, interior portions of the housing 12 can be shaped to retain the outer annular support within the housing 12. For example, as shown in FIG. 2B, as well as in FIGS. 3A and 3B, the sidewall 18 of the housing 12 can comprise an annular inwardly sloping tapered portion 28. Similarly, an outer surface of the outer absorbent support 20 can include an angled portion 30 matching the tapered portion 28 of the sidewall 18. The interface or friction engagement between the tapered portion 28 of the sidewall 18 and angled portion 30 of the housing 12 can be configured to ensure that the outer absorbent support 20 remains seated in the housing 12. In particular the interface or friction engagement can ensure that the outer absorbent support 20 does not lift away from the housing 12 when the male connector 110 is inserted into or removed from the housing 12.

In some examples, the housing 12 further comprises protrusions, such as axially extending ribs or ridges 32, extending outward from an outer surface of the sidewall 18 of the housing 12. The axial ridges 32 can be provided to increase rigidity of the housing 12 compared to if ridges 32 were not present. Also, the ridges 32 can make the housing 12 easier to grasp and manipulate improving usability of the cap 10 and, for example, making it less likely that the practitioner will drop or mishandle the cap 10 during use.

As shown most clearly in FIGS. 2B and 2C, the cap 10 further comprises the outer absorbent support 20 disposed within the housing 12, which can also be referred to as an absorber, absorbent member, cushion, padding, or sponge. As described in further detail herein, the outer absorbent support 20 is a flexible, deformable, or compressible structure configured to compress axially when the male connector 110 is inserted into the housing 12. In particular, the outer absorbent support 20 is configured to compress so that the annular shield 122 of the male connector 110 can be inserted farther into the housing 12 allowing threads 124 of the shield 122 to come into contact with an engage the socket 24. In some examples, the outer absorbent support 20 can be an annular or tubular structure defining the recess 22 having an outer diameter that substantially matches (e.g., is the same as or within about 5% or less of) an inner diameter of the housing 12. For example, the outer absorbent support 20 can have a top portion with an outer diameter OD1 (shown in FIG. 2B) matching an inner diameter ID1 (shown in FIG. 3B) of the housing 12 and a bottom portion having an outer diameter OD2 (shown in FIG. 2B) matching an inner diameter ID2 (shown in FIG. 3B) of the housing 12. In some examples, the recess 22 can be an irregular shape, such as a recess or cavity formed from different sized connected cylindrical and/or elongated cavities. For example, the recess 22 can include a first section 22a or portion having a diameter ID3 (shown in FIG. 2B) corresponding to an outer diameter of an annular shield 122 of the male connector 110 and a second section 22b or portion axially aligned with the first portion 22a having a diameter ID4 (shown in FIG. 2B) corresponding to an outer diameter of the socket 24. As shown in FIG. 2B, the diameter ID3 of the first section 22a can be wider than the diameter ID4 of the second section 22b.

The outer absorbent support 20 can be formed from a flexible and/or compressible material that deforms and presses against portions of the male connector 110 as the male connector 110 is inserted into the housing 12. For example, the outer absorbent support 20 can be configured to axially compress as the male connector 110 is inserted to the housing 12. Also, the outer absorbent support 20 can be configured to deform or bulge radially inwardly pressing against surfaces of the male connector 110. For example, the outer absorbent support 20 can press against a distal end of the annular shield 122 and against an outer surface of the annular shield 122 to seal portions of the male connector 110, which prevents contamination of inner spaces enclosed by the annular shield 122. Also, the outer absorbent support 20 can be configured to press against the outer surface of the annular shield 112 pushing into any gaps, slots, cut-away portions, channels, cavities, grooves, recessed areas, or any other spaces on the outer surface of the annular shield 122, which eliminates any spaces where contaminants, such as microbes, dirt, particles, or other debris, may collect. Accordingly, the deformable outer absorbent support 20 contributes to forming a tight and secure connection between surfaces of the male connector 110 and interior component of the cap 10, thereby contributing to the cleaning, disinfecting, and contamination avoiding features of the cap 10.

In some examples, the outer absorbent support 20 comprises a thermoplastic elastomer, such as polypropylene, polyethylene, or synthetic or natural rubber (e.g., isoprene). The outer absorbent support 20 can also comprise a porous foam (e.g., an open cell foam) or sponge capable of absorbing the cleaning or disinfecting solution, such as a foam or sponge comprising polyurethane. In other examples, the foam material can be a Plastazote® foam, which is an engineered polymer foam by Zotefoams PCL.

The cap 10 further comprises the socket 24, which engages the male connector 110, thereby securing the male connector 110 within the housing 12. As shown in FIGS. 2B and 2C, the socket 24 can be a tubular member secured or adhered to the housing 12 comprising an open first end or top 34, a second end or bottom 36, and a sidewall 38 extending between the top 34 and the bottom 36. For example, as shown in FIG. 2B, the socket 34 can include a closed bottom 36 having an outer surface that is adhered to an inner surface of the bottom 16 of the housing 12. The socket 34 can be entirely enclosed within the housing 12 meaning, for example, that the open top 34 of the socket 24 is recessed relative to the open top 14 of the housing 12 by a distance D1 (shown in FIG. 2B).

The socket 24 generally is formed from or comprises a rigid thermoplastic polymer material, such as the same material used to form the housing 12. For example, the socket 24 can be formed from a rigid polymer material, such as polyester, polycarbonate, polypropylene, polyethylene, polyethylene terephthalate, or acrylonitrile butadiene styrene.

In some examples, the socket 24 further comprises a thread 40 extending radially outward from the sidewall 38 of the socket 24. The thread 40 can be configured to engage threads 124 on an inner surface 126 of the annular shield 122 of the male connector 110 for securing the male connector 110 to the socket 24. Accordingly, the male connector 110 can be securely retained within the housing 12 by the threaded engagement preventing the cap 10 from being mistakenly removed from the male connector 110 at unexpected or inappropriate times.

In some examples, the socket 24 further comprises one or more openings in the sidewall of the socket 24 for permitting liquid, such as cleaning solution, to pass from an interior of the socket 24 to the outer absorbent support 20 surrounding the socket 24. For example, as shown in FIGS. 2B and 2C, the opening can be a slot 42 extending axially from the open top 34 of the socket 24. In some examples, as shown in the figures, the socket 24 can include opposing slots 42 on opposite sides of the socket 24 for providing cleaning solution to different areas or regions of the outer absorbent support 20. In other examples, the openings can be holes (e.g., square, rectangular, circular, or elliptical holes), slits, perforations, or any other openings extending through the sidewall 38 of the socket 24 for permitting the cleaning solution to pass from the interior of the socket 24 to the outer absorbent support 20.

The cap 10 further comprises the inner absorbent support 26 disposed within the socket 24. As previously described, the inner absorbent support 26 contains the cleaning solution for cleaning and/or disinfecting portions of the male connector 110 engaged to the cap 10. As shown in FIG. 2B, the inner absorbent support 26 can be disposed and held within a cylindrical interior space or cavity defined by the socket 24. For example, the inner absorbent support 26 can be held in place within the socket 24 by a conventional adhesive or mechanical fastener. In other examples, the inner absorbent support 26 can be held in place in the socket 24 by friction between an inner surface of the socket 24 and an outer surface of the inner absorbent support 26.

The inner absorbent support 26 can be formed from the same material as the outer absorbent support 20, specifically from a material that is capable of absorbing the cleaning solution and expelling the cleaning solution from the inner absorbent support 26 when compressed. For example, the inner absorbent support 26 can be formed from a thermoplastic elastomer, such as polypropylene, polyethylene, or synthetic or natural rubber (e.g., isoprene). The inner absorbent support 26 can also comprise a porous foam (e.g., an open cell foam) or sponge capable of absorbing the cleaning or disinfecting solution, such as a foam or sponge comprising polyurethane. In other examples, the foam material can be a Plastazote® foam, which is an engineered polymer foam by Zotefoams PCL.

In some examples, the inner absorbent support 26 can be provided (i.e., presoaked) with the cleaning or disinfecting solution. For example, the cleaning or disinfecting solution can be an antimicrobial, anti-fungal, antibacterial, or antiviral solution that cleans and sterilizes surfaces of the male connector 110. In some examples, the cleaning solution can be isopropyl alcohol (IPA), such as about 70% IPA. In other examples, the cleaning solution can be about 0.5% to about 3.5% chlorhexidine gluconate in combination with about 70% IPA. A chlorohexidine composition may be beneficial because it has a slower evaporation rate than IPA and, therefore, provides a more persistent disinfectant activity after the cap 10 is removed from the male connector 110 and before the VAD is connected to the hub, port, or valve.

The inner absorbent support 26 can be configured to axially compress as the stem 114 of the male connector 110 is inserted into and moves through the interior of the socket 24. Axial compression of the inner absorbent support 26 expels the cleaning solution from the inner absorbent support 26. The expelled cleaning solution moves through the interior of the socket 24 towards the open top 34 of the socket 24 contacting an outer surface of the stem 114 of the male connector 110 inserted into the socket 24. Once expelled from the inner absorbent support 26, the cleaning solution can also pass through openings, such as the slots 42, of the socket 24. After passing through the slots 42, the cleaning solution comes into contact with other parts of the male connector 110, such as the threads 124, inner surface 126, and/or outer surface of the annular shield 122. The cleaning solution can also be absorbed by the outer absorbent support 20, such that cleaning solution remains in proximity to surfaces of the annular shield 122 for cleaning and disinfecting these surfaces.

In some examples, the cap 10 further comprises a seal 44 positioned in the socket 24 over the inner absorbent support 26 (e.g., positioned between a top of the inner absorbent support 26 and the top 34 of the socket 24). The seal 44 can prevent fluids, such as cleaning or disinfecting solution absorbed by the inner absorbent support 26, from flowing into the lumen enclosed by the stem 114 of the male luer connector 110. In some examples, the seal 44 can be formed from a thermoplastic elastomer comprising, for example, an elastomeric closed cell foam. Also, the seal 44 can comprise an abrasive material that is capable of scrubbing or mechanically removing objects, such as microbes, dirt, particles, and other debris, from a distal surface of the stem 114, while, at the same time, limiting ingress of the cleaning solution into the lumen of the stem 114.

In some examples, the seal 44 includes a layer 46 of the cleaning solution over a top surface of the seal 44. The layer 46 of cleaning solution can be provided to clean and disinfect the distal end of the stem 114 of the male connector 110, contributing to the cleaning and disinfecting functionality provided by the cap 10.

The seal 44 is sized and positioned so that cleaning solution expelled from the inner absorbent member 26 can move past the seal 44 towards the top 34 of the socket 24 and/or the top 14 of the housing 12. As previously discussed, as the cleaning solution moves through the interior of the socket 24, the cleaning solution contacts the outer surface of the stem 114 for cleaning and disinfecting the outer surface. In order for the cleaning solution to move past the seal 44, an outer diameter of the seal 44 can be smaller than either an inner diameter of the socket 24 or an outer diameter of the inner annular support 26 so that the seal 44 does not prevent the cleaning solution from moving towards the top 34 of the socket 24 and coming into contact with the outer surface of the stem 114. In other examples, the seal 44 can include holes, cut-away portions, grooves, slits, slots, and/or other openings for permitting the cleaning solution to move through the seal 44 towards the top 34 of the stem 24.

In some examples, as shown in FIG. 2A, the cap 10 can further comprise a removable and/or disposable protective cover 48 positioned over the open top 14 of the housing 12. The protective cover 48 can be provided to protect components and portions of the cap 10, such as the housing 12, outer absorbent support 20, socket 24, inner absorbent support 26, and seal 44, during transport and storage, as well as to prevent contamination and to prevent the cleaning or disinfecting solution from evaporating prior to use. The protective cover 48 can comprise a sheet, such as a polymer film, with adhesive on a first side of the sheet for removably mounting the protective cover 48 to the open top 14 of the housing 12. Alternatively, the protective cover 48 can be removably mounted to the open top 14 of the housing 12 by heat sealing. The protective cover 42 can be formed from a material that is impervious or substantially impervious to air, so that the layer 46 of cleaning solution on the top surface of the seal 44 and/or cleaning solution absorbed by the inner absorbent support 26 does not evaporate or dry-out prior to use of the cap 10. Accordingly, the protective cover 48 can increase a shelf life of the cap 10, as well as prevent microbes and other debris from collecting in the cap 10 prior to use.

### Method for Attaching a Connector to the Cap

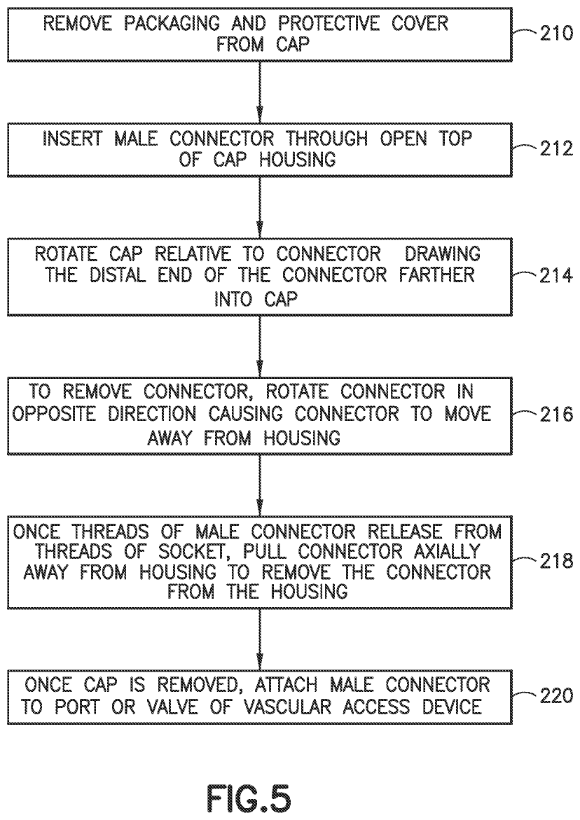

As previously described, the cap 10 of the present disclosure is a disinfecting cap 10 configured to be connected to various types and sizes of male connector 110. FIGS. 4A-4C show the cap 10 being connected to a male connector 110. FIG. 5 is a flow chart showing a method for connecting and/or disconnecting the disinfecting cap 10 from the male connector 110.

As shown in FIG. 5, at step 210, in order to connect the cap 10 to a male connector 110, the practitioner first removes any packaging from the cap 10 and removes the protective cover 48 from the open top 14 of the housing 12.

At step 212, once the packaging and protective cover 48 are removed, the practitioner moves the connector 110 towards the open top 14 of the housing 12 in a direction of arrow A1 (shown in FIG. 4A). Specifically, the stem 114 of the male connector 110 is inserted through the open top 14 of the housing 12 and through the open top 34 of the socket 24 causing the distal end of the stem 114 of the male connector 110 to contact the seal 44 disposed in the socket 24, as shown in FIG. 4B. As previously described, the layer 46 of cleaning solution on the seal 44 can be provided to disinfect the distal end of the stem 114. Also, contact between the distal end of the stem 114 and the seal 44 can seal off the lumen extending through the stem 114 preventing cleaning solution or other fluids from passing into the lumen of the male connector 110. Contact between the distal end of the stem 114 and the seal 44 can also mechanically remove particles, such as microbes, dirt, and other debris, from the male connector 110. Moving the male connector 110 towards the cap 10 also brings the annular shield 122 of the male connector 110 into contact with the socket 24. Specifically, as shown in FIG. 4B, the threads 124 of the shield 122 are brought into contact with the thread 40 on the outer surface of the socket 24.

At step 214, in order to move the male connector 110 farther into the housing 12 of the cap 10, the practitioner next rotates the male connector 110 relative to the cap 10 in a direction of arrow A2 (shown in FIG. 4B). Rotation of the male connector 110 relative to the housing 12 and socket 24 draws the male connector 110 farther into the cap 10, as shown by arrow A3 in FIG. 4B, causing the distal end of the stem 114 to move through the interior of the socket 24. As the stem 114 moves through the socket 24, the inner absorbent support 26 axially compresses expelling the cleaning solution from the inner absorbent support 26 into the interior of the socket 24. The cleaning solution moves from the inner absorbent support 26, past the seal 44, and towards the open top 34 of the socket 24. As the cleaning solution is pushed through the socket 24, the cleaning solution contacts the outer surface of the stem 114 cleaning and disinfecting the outer surface of the stem 114. The cleaning solution then passes through the slots 42 in the sidewall 38 of the socket 24 and is absorbed by the outer absorbent support 20.

Rotation of the male connector 110 relative to the housing 12 also causes the annular shield 122 to move into the top section 22a of the recess 22 defined by the outer absorbent support 20. Continued movement of the annular shield 122 into the housing 12 causes the shield 122 to press against the outer absorbent support 20, which compresses the outer absorbent support 20. As previously described, the outer absorbent support 20 can also bulge radially inward pushing into gaps, slots, cut-away portions, channels, cavities, grooves, or any other spaces on the outer surface of the annular shield 122, which eliminates gaps or open spaces where contaminants, such as microbes, dirt, or other debris, may collect. As previously described, cleaning solution from the inner absorbent support 26 can also pass towards the threads 124 of the annular shield 122 through the slots 42 of the socket 24 for cleaning and disinfecting the threads 124 of the annular shield 122. The cleaning solution can also be absorbed by the outer absorbent support 20, such that cleaning solution remains in close proximity to surfaces of the annular shield 122, thereby protecting the inner and outer surfaces of the annular shield 122 from contamination.

At step 216, in order to remove the cap 10 from the male connector 110, the practitioner first rotates the male connector 110 relative to the housing 12 and socket 24 in a direction opposite from direction A2 (shown in FIG. 4B) causing the threads 124 of the annular shield 122 to back off of the thread 40 on the outer surface of the socket 24. The rotation of the male connector 110 relative to the socket 24 causes the male connector 110 to begin to move axially out of the housing 12.

At step 218, once the threads 124 of the male connector 110 are released from the thread 40 of the socket 24, the practitioner can pull the male connector 110 away from the housing 12, as shown by arrows A4 in FIG. 4C, thereby releasing the male connector 110 from the cap 10. For example, the practitioner can grasp the housing 12 of the cap 10 with one hand and the male connector 110 with the other hand. The practitioner then pulls the male connector 110 away from the cap 10 to remove the male connector 110 from the cap 10. Once removed, the cap 10 can be discarded, as it is often a disposable single-use product.

Once the male connector 110 is fully removed from the housing 12, at step 220, the male connector 110 can be connected to a VAD. For example, the male connector 110 can be attached or inserted into a female hub, port, or valve of the VAD forming a needleless fluid-tight connection between the male connector 110 and a fluid path, channel, or lumen of the VAD.

Disinfecting Cap with Multi-Part Housing

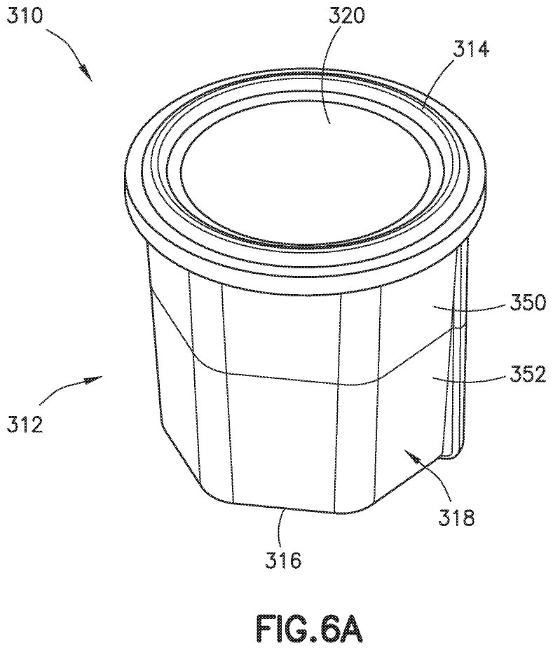

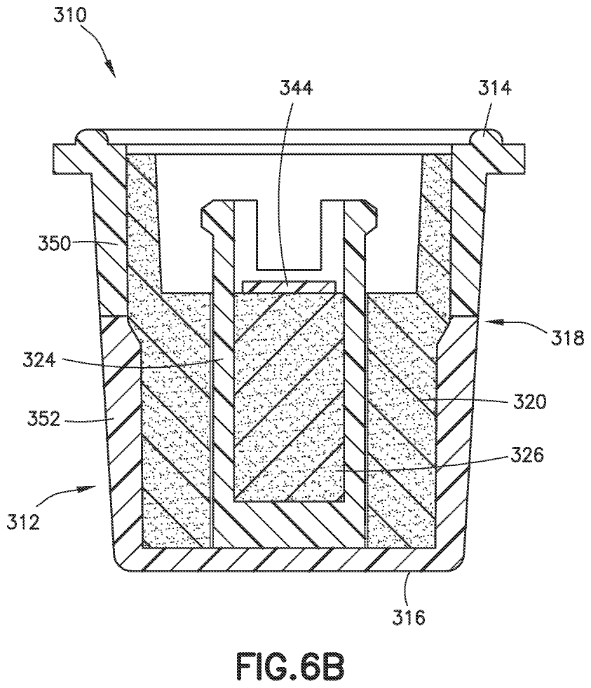

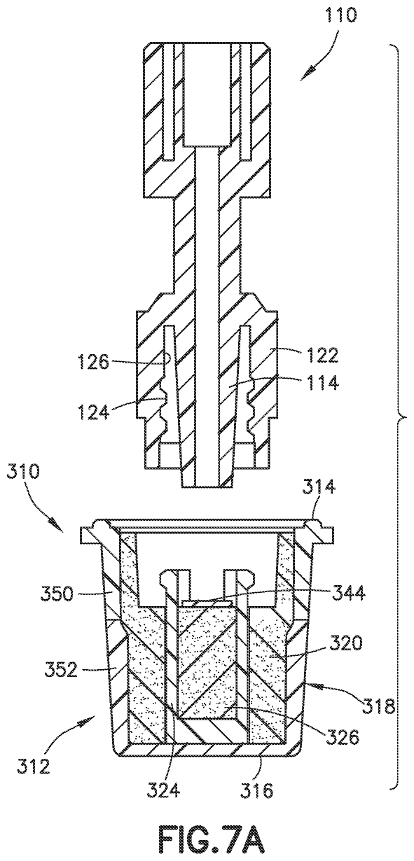

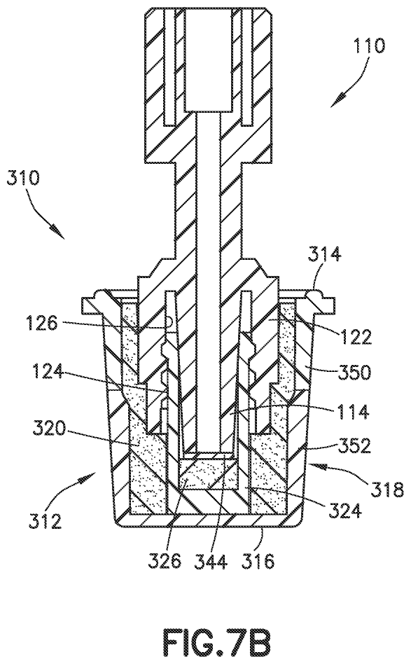

FIGS. 6A-7B show another exemplary disinfecting cap 310 for a male connector 110 including features of the present disclosure. Specifically, FIG. 6A shows a perspective view of the cap 310 and FIG. 6B shows a cross-sectional view of the cap 310. FIG. 7A shows a cross-sectional view of the cap 310 with a male connector 110, prior to inserting the male connector 110 into the cap 310. FIG. 7B shows the male connector 110 fully inserted into the cap 310.

As in previous examples, the cap 310 includes a housing 312 having an open first end or top 314, a second end or bottom 316, and a sidewall 318 extending between the top 314 and the bottom 316. The cap 310 also includes the outer absorbent support 320 disposed in the housing 312 defining the central recess and the socket 324 positioned in the recess. Also, the cap 310 comprises the seal 344 and the inner absorbent support 326 disposed in the socket 324 configured to contain the cleaning solution for cleaning and/or disinfecting portions of the male connector 110 engaged to the cap 310.

The cap 310 differs from previous examples in that the housing 312 is formed from multiple parts. Specifically, the housing 312 comprises a first part 350, which comprises the open top 314 of the housing 312. For example, the first part 350 can be a tubular member comprising an open top, an open bottom, and a sidewall extending between the top and the bottom. The first part 350 of the housing 312 is formed from a flexible material, such as a thermoplastic elastomer, such as silicone, polypropylene, polyethylene, or synthetic or natural rubber (e.g., isoprene). Further, the first part 350 can be formed from a flexible material having a shore hardness A value less than or equal to 130 (Shore A). Beneficially, the flexible first part 350 of the housing 310 can be capable of stretching, bending, or otherwise deforming so that the cap 310 can engage different sizes and shapes of male connectors 110.

The housing 310 also includes a second part 352, which includes the closed bottom 316 of the housing 312. The second part 352 can comprise an open top adhered to the open bottom of the first part 350, a closed bottom, and a sidewall extending between the top and the bottom. The second part 352 is formed from a rigid material, such as a rigid thermoplastic polymer. For example, the second part 352 can be formed from one or more of polyester, polycarbonate, polypropylene, polyethylene, polyethylene terephthalate, or acrylonitrile butadiene styrene. Further, the second part 352 can be formed from a rigid and durable material, such as a material having a shore hardness D value of less than or equal to 95 (Shore D).

In some examples, both parts 350, 352 of the housing 312 can be formed by over-molding of other components of the cap 310. For example, the second part 352 can be formed by over-molding of the rigid thermoplastic polymer about a bottom portion of the outer absorbent support 352 and socket 324. After the rigid thermoplastic polymer dries or cures, the first part 350 can be formed about the top portion of the outer absorbent support 320 by an over-molding method, which adheres the first part 350 to the outer absorbent support 320 and to the previously molded second part 352.

Cap with Holding Knob

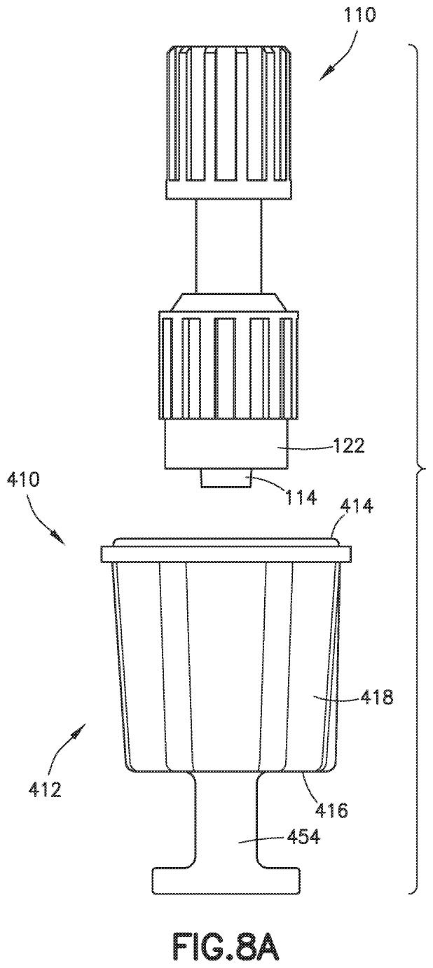

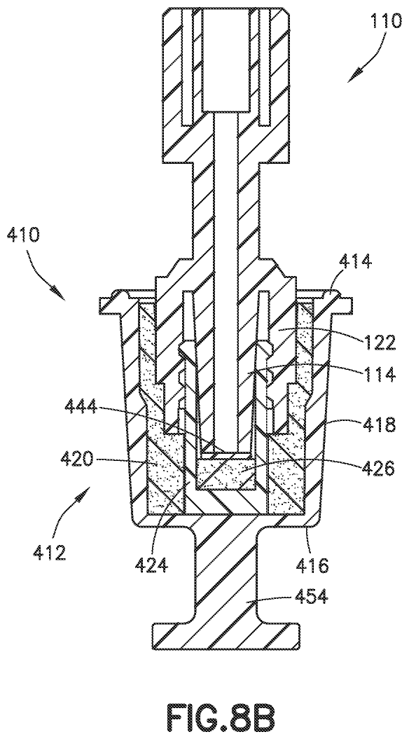

FIGS. 8A and 8B show another example of a cap 410 that includes features of the present disclosure. Specifically, FIG. 8A is a front view showing the male connector 110 and the cap 410, prior to insertion of the male connector 110 into the cap 410. FIG. 4B is a cross-sectional view showing the male connector 110 fully inserted into the cap 410. As in previous examples, the cap 410 comprises the housing 412 comprising the open first end or top 414, the closed second end or bottom 416, and the sidewall 418 extending between the top 414 and the bottom 416. The cap 410 also includes the outer absorbent support 420 disposed in the housing 412 defining the central cavity or recess and the socket 424 disposed in the recess. The socket 424 is configured to engage the male connector 110 for retaining the male connector 110 within the housing 412. The cap 410 also comprises the seal 444 and the inner absorbent support 426 disposed in the socket 424 configured to contain the cleaning solution for cleaning and/or disinfecting portions of the male connector 110 engaged to the cap 410.

The cap 410 differs from previous examples in that the cap further comprises a knob 454 extending from the bottom 416 of the housing 412. The knob 454 can be integrally formed with the bottom 416 of the housing 412 or can be adhered to the housing 412 by, for example, ultrasonic welding or using conventional fasteners or adhesives. Both the housing 412 and the knob 454 can be formed from a rigid thermoplastic polymer, such as such as polyester, polycarbonate, polypropylene, polyethylene, polyethylene terephthalate, or acrylonitrile butadiene styrene.

As shown in FIGS. 8A and 8B, the knob 454 can be T-shaped including a vertical leg and a horizontal part attached to the leg. The knob 454 can be configured to be grasped by the practitioner to facilitating manipulating the cap 10 to connect the cap 410 to and to disconnect the cap 410 from the male connector 110. For example, the practitioner can grasp the knob 454 to rotate the cap 410 about the male connector 110. Also, the practitioner can grasp the knob 454 to pull the cap 410 away from the male connector 110, thereby removing the cap 410 from the male connector 110.

### Alternative Seal Configurations for Disinfecting Cap

As previously described, the caps 10, 310, 410 disclosed herein can include the seals 44, 344, 444 positioned between the inner absorbent support 26, 326, 426 and the open top 34 of the socket 24, 324, 424. Further, as previously described, the seal 44, 344, 444 can be positioned so that the cleaning solution can move upwards through the socket 24, 324, 424 in order to contact and wet the outer surface of the stem 114. In some examples, an outer diameter of the seal 44, 344, 444 can be smaller than an inner diameter of the socket 24, 324, 444 to allow the cleaning solution to move past the seal 44, 344, 444. In other examples, the seal 44, 344, 444 can include various holes, slits, perforations, or other openings for permitting the upwards movement of the cleaning solution through the interior of the socket 24, 324, 424.

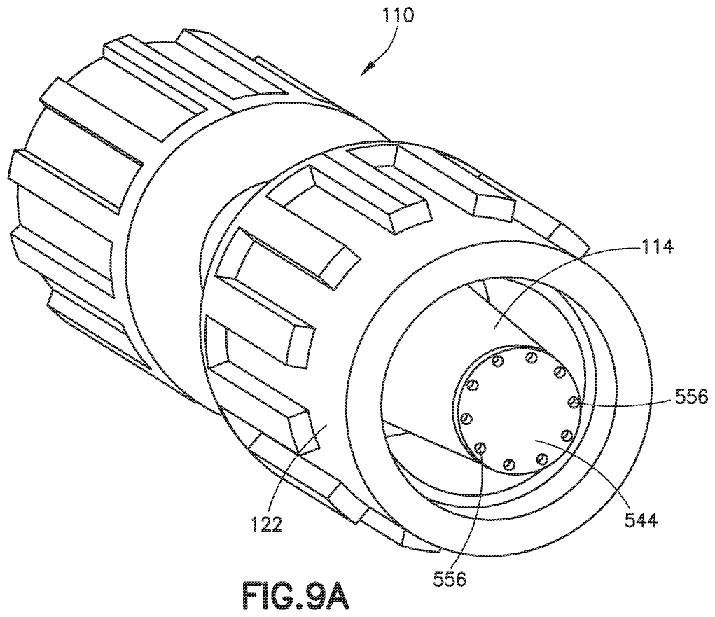

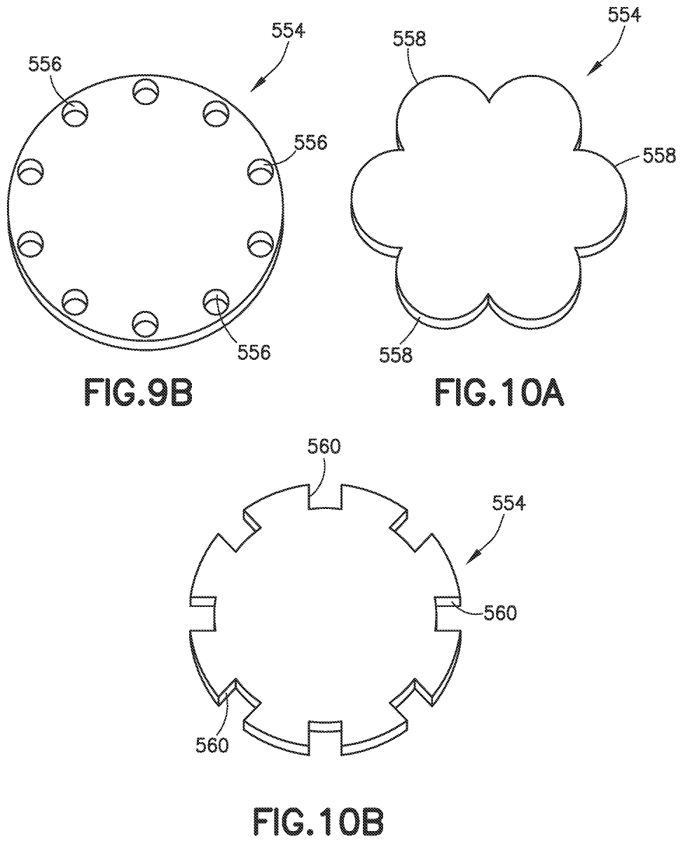

FIGS. 9A-10B show different seal 544 configurations that can be used for permitting the upwards movement of cleaning solution through the socket 24, 324, 424. Specifically, FIG. 9A shows a male connector 110 with a seal 544 positioned over the distal end of the stem 114 to seal the lumen extending through the stem 114. FIG. 9B is an enlarged perspective view of the seal 544. As shown in FIGS. 9A and 9B, the seal 544 comprises circular holes 556 positioned about the periphery of the seal 544 for permitting the cleaning solution to move through seal 544 and through the interior of the socket 24, 324, 424 to contact the outer surface of the stem 114, as previously described. As shown in FIGS. 9A and 9B, the seal 544 includes ten identical circular openings 556 equidistantly spaced about the periphery of the seal. However, the number, distribution, and shape of the openings 556 shown in FIGS. 9A and 9B is not intended to be limiting. In other examples, the seal 544 may include fewer than ten openings 556 or more than ten openings 556. Also, the openings 556 may be the same size and shape or may be different sizes and shapes. Further, the openings 556 could be irregularly spaced about the periphery of the seal 544 in order to provide more cleaning solution to some portions of the male connector 110 and less cleaning solution to other portions of the male connector 110.

FIGS. 10A and 10B show additional configurations of seals 544 that can also be used with the caps 10, 310, 410 of the present disclosure. For example, the seal 544 of FIG. 10A includes multiple lobes 558 extending about the periphery of the seal 544 so that the cleaning solution can move past the seal 544 and through the socket 24, 324, 424 of the cap 10, 310, 410. In a similar manner, FIG. 10B shows a seal 544 with cut-outs 560 positioned to permit the cleaning solution to move past the seal 544. The seal configurations shown in FIGS. 9A-10B are not intended to be limiting of the present disclose and other configures for the seals 544 may be determined by those skilled in the art including various combinations of the holes or openings 556, lobes 558, and/or cut-outs 560 shown in FIGS. 9A-10B, as well various other shapes for openings or cut away portions for permitting the cleaning solution to move past the seal 544.

While examples of the disinfecting caps 10, 310, 410 and methods of use of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A cap configured to engage a male connector, the cap comprising:
a housing comprising an open first end, a second end, and at least one sidewall extending between the first end and the second end;
an outer absorbent support disposed in the housing defining a recess;
a socket disposed in the recess of the outer absorbent support configured to engage the male connector for retaining the male connector within the housing; and
an inner absorbent support disposed in the socket configured to contain a cleaning solution for cleaning and/or disinfecting portions of the male connector engaged to the cap.

2. The cap of claim 1, wherein the male connector comprises a luer stem defining a central lumen and a shield extending about the stem, and
wherein the shield of the male luer connector comprises threads on an inner surface of the shield configured to engage corresponding threads of a female luer connector for securing the male connector to the female luer connector.

3. The cap of claim 2, wherein, when the male connector is engaged to the cap, the luer stem of the male connector is inserted into the socket and an outer surface of the annular shield is in contact with an inner surface of the outer absorbent support.

4. The cap of claim 2, wherein, when the male connector is engaged to the cap, cleaning solution is exposed to the threads on the inner surface of the annular shield for cleaning and/or disinfecting the threads.

5. The cap of claim 1, wherein an inner surface of the sidewall of the housing comprises an inwardly sloping tapered portion, and wherein an outer surface of the outer absorbent support comprises an angled portion matching the tapered portion of the sidewall.

6. The cap of claim 1, wherein the housing comprises a first part, which comprises the open first end of the housing, formed from a flexible material, and a second part, which comprises the closed second end of the housing, formed from a rigid material.

7. The cap of claim 6, wherein the first material comprises a thermoplastic elastomer comprising at least one of silicone, polypropylene, polyethylene, or synthetic or natural rubber, and the second material comprises a rigid thermoplastic polymer, comprising at least one of polyester, polycarbonate, polypropylene, polyethylene, polyethylene terephthalate, or acrylonitrile butadiene styrene.

8. The cap of claim 1, further comprising a knob extending from the second end of the housing for grasping the housing.

9. The cap of claim 1, wherein the recess defined by the outer absorbent support comprises a first portion having a first diameter corresponding to an outer diameter of an annular shield of the male connector and a second portion axially aligned with the first portion having a second diameter corresponding to an outer diameter of the socket.

10. The cap of claim 1, wherein the socket comprises an open first end, a second end, and a sidewall extending between the first end and the second end, and
wherein the socket further comprises a thread extending radially outward from the sidewall of the socket, the thread configured to engage threads on an inner surface of a shield of the male connector.

11. The cap of claim 10, wherein the engagement between the thread of the socket and the threads of the shield of the male connector rotatably secures the male connector within the housing.

12. The cap of claim 10, wherein the sidewall of the socket comprises at least one opening for permitting the cleaning solution to pass through the socket to the outer absorbent support.

13. The cap of claim 12, wherein the at least one opening comprises a first slot extending axially from the open first end of the socket and a second slot extending axially from the open first end of the socket on an opposite side of the socket from the first slot.

14. The cap of claim 1, wherein insertion of the male connector into the socket causes the inner absorbent support to axially compress farther into the socket, and
wherein the axial compression of the inner absorbent support expels the cleaning solution from the inner absorbent support causing the cleaning solution to move through an interior of the socket contacting an outer surface of a stem of the male connector inserted in the socket.

15. The cap of claim 1, further comprising the cleaning solution absorbed by the inner absorbent support, wherein the cleaning solution comprises Isopropyl Alcohol (IPA) and/or a solution comprising from about 0.5% to about 3.5% chlorhexidine gluconate and about 70% IPA

16. The cap of claim 1, further comprising a seal disposed in the socket over the inner absorbent support configured to seal an open end of the male connector, thereby preventing the cleaning solution from entering a lumen of the male connector,
wherein the seal is positioned to permit the cleaning solution expelled from the inner absorbent member to move past the seal towards the first end of the housing when the inner absorbent member is axially compressed.

17. The cap of claim 16, wherein the seal comprises at least one of holes, cut-away portions, grooves, slits, or slots for permitting the cleaning solution to move past the seal towards the open first end of the housing.

18. The cap of claim 16, further comprising a layer of the cleaning solution over a top surface of the seal.

19. A method for attaching the cap of claim 1 to the male connector, the method comprising:
inserting a distal end of a stem of the male connector through the open first end of the housing causing the distal end of the stem to contact a seal disposed in the socket over the inner absorbent support; and
rotating the male connector relative to the housing causing the stem of the male connector to advance through the socket axially compressing the inner absorbent support.

20. A manufacturing method for the cap of claim 1, the method comprising:
inserting the inner absorbent support into the socket;
inserting the socket and inner absorbent support inserted therein into the recess of the outer absorbent support; and
forming the housing about an outer surface of the outer absorbent support by over-molding.
